(19) United States
(12) Patent Application Publication
(10) Pub. No.: US 2024/0350301 A1
(43) Pub. Date: Oct. 24, 2024

(54) THERMAL INTRAVENOUS SLEEVE

(71) Applicant: NEA TIVS, LLC, Scottsdale, AZ (US)

(72) Inventors: Nicholas Andrisevic, Scottsdale, AZ (US); Emily Andrisevic, Scottsdale, AZ (US)

(21) Appl. No.: 18/644,833

(22) Filed: Apr. 24, 2024

Related U.S. Application Data
(60) Provisional application No. 63/461,419, filed on Apr. 24, 2023.
(60) Provisional application No. 63/553,911, filed on Feb. 15, 2024.

(51) Int. Cl. A61F 7/00; A61B 17/00; A61B 17/132

(52) U.S. Cl. CPC A61F 7/007; A61B 17/1322; A61B 2017/00862; A61F 2007/0031; A61F 2007/0032; A61F 2007/0035; A61F 2007/0086

(57) ABSTRACT

Embodiments of the present disclosure can include a thermal intravenous sleeve. The thermal intravenous sleeve can include a thermally insulative material, wherein the thermally insulative material includes a proximal end, a distal end, first lateral end, and second lateral end, wherein a portion of the first lateral end and the second lateral end are joined, thereby defining an interior sleeve portion and an exterior sleeve portion. In some embodiments, the thermal intravenous sleeve can include a thermal element disposed within the interior sleeve portion. In some embodiments, a tourniquet can be disposed at a proximal end of the thermal intravenous sleeve.

### CROSS-REFERENCE TO RELATED APPLICATIONS

This application claims the benefit of U.S. provisional application No. 63/461,419, filed 24 Apr. 2023 (the '419 application). This application claims the benefit of U.S. provisional application No. 63/553,911, filed 15 Feb. 2024 (the '911 application). The '419 application and the '911 application are both hereby incorporated by reference in their entirety as though fully set forth herein.

### A. FIELD OF THE DISCLOSURE

This disclosure relates to a thermal intravenous sleeve.

### B. BACKGROUND ART

Approximately 200 million peripheral intravenous (IV) catheters are started annually, with 80 percent of patients that are hospitalized having a peripheral intravenous catheter. Additionally, between 7 to 14 billion clinical lab tests are performed each year in over 300,000 laboratories. Numerous conditions can lead to difficulty in starting IV catheters, including obesity, hypovolemia, anatomical variations, drug use, and chronic medical problems. As a result of these conditions, a failure rate associated with placement of IV catheters can be around 34%, which can lead to increased nursing time; patient dissatisfaction due to multiple IV attempts; and/or extra equipment costs.

In medical studies, heat has been used to warm a patient in order to increase a success rate for placement of an IV. For example, heat can be used to warm an approximate location of a patient where an IV will be placed, using a heat pack, electric heating pad, dry/warm blankets, etc. As a result of the use of heat, a success rate of IV placement has been shown to increase from 66% without heat to 92% with heat.

### BRIEF SUMMARY

Embodiments of the present disclosure can include a thermal intravenous sleeve. The thermal intravenous sleeve can include a thermally insulative material, wherein the thermally insulative material includes a proximal end, a distal end, first lateral end, and second lateral end, wherein a portion of the first lateral end and the second lateral end are joined, thereby defining an interior sleeve portion and an exterior sleeve portion. In some embodiments, the thermal intravenous sleeve can include a thermal element disposed within the interior sleeve portion. In some embodiments, a tourniquet can be disposed at a proximal end of the thermal intravenous sleeve.

Embodiments of the present disclosure can include a thermal intravenous sleeve. The thermal intravenous sleeve can include a thermally insulative material, wherein the thermally insulative material includes a proximal end, a distal end, first lateral end, and second lateral end, wherein a portion of the first lateral end and the second lateral end are joined, thereby defining an interior sleeve portion and an exterior sleeve portion. In some embodiments, an electrical heating element disposed within the interior sleeve portion. In some embodiments, an integrated tourniquet cuff disposed at a proximal end of the thermal intravenous sleeve.

Embodiments of the present disclosure can include a thermal intravenous sleeve. The thermal intravenous sleeve can include a thermally insulative material, wherein the thermally insulative material includes a proximal end, a distal end, first lateral end, and second lateral end, wherein a portion of the first lateral end and the second lateral end are joined, thereby defining an interior sleeve portion and an exterior sleeve portion. In some embodiments, a pocket can be disposed on the interior sleeve portion. In some embodiments, a chemical heating element can be disposed in the pocket. In some embodiments, a tourniquet can be disposed at a proximal end of the thermal intravenous sleeve.

### BRIEF DESCRIPTION OF THE DRAWINGS

FIG. 1A is a top view of a thermal intravenous sleeve disposed on the arm of a patient, in accordance with embodiments of the present disclosure.

FIG. 1B is a top view of the thermal intravenous sleeve depicted in FIG. 1A, in an opened configuration, in accordance with embodiments of the present disclosure.

FIG. 1C is a top and side view of a thermal intravenous sleeve in FIGS. 1A and 1B, in an opened configuration with the arm of a patient inserted therein, in accordance with embodiments of the present disclosure.

FIG. 2A is a top view of a thermal intravenous sleeve with an integrated tourniquet cuff disposed on the arm of a patient, in accordance with embodiments of the present disclosure.

FIG. 2B is a top view of the thermal intravenous sleeve depicted in FIG. 2A, in an opened configuration, in accordance with embodiments of the present disclosure.

FIG. 3A is a side view of a thermal intravenous sleeve that includes an electrical heating element, in accordance with embodiments of the present disclosure.

FIG. 3B is a top view of the thermal intravenous sleeve depicted in FIG. 3A, in accordance with embodiments of the present disclosure.

FIG. 4A depicts a graph depicting test results associated with embodiments of the present disclosure versus known methods, in accordance with embodiments of the present disclosure.

FIG. 4B depicts a table depicting test results associated with embodiments of the present disclosure versus known methods, in accordance with embodiments of the present disclosure.

### DETAILED DESCRIPTION

In some embodiments of the present disclosure, the thermal intravenous sleeve can be made from a thermally insulative material and can include a thermal element that can be used to warm a patient's forearm, in order to improve the success rate at which a peripheral intravenous or peripherally inserted central catheter can be placed in the patient. In use, a patient's arm can be inserted into the thermal intravenous sleeve for a period of time (e.g., 5 minutes) and a thermal element can be activated, thereby allowing the patient's forearm and/or portions of the patient's forearm to be warmed. Application of heat is known to improve a success rate at which peripheral IVs can be placed. Embodiments of the present disclosure can increase an effectiveness at which peripheral IVs can be placed, over other methods, such as application of a heat pack, an electric heating pad, a dry/wet warm blanket. For example, embodiments of the present disclosure can provide for higher achieved temperature averages on a patient's arm and faster times to reach maximum temperature. This can result in an increased case of and faster intravenous insertion.

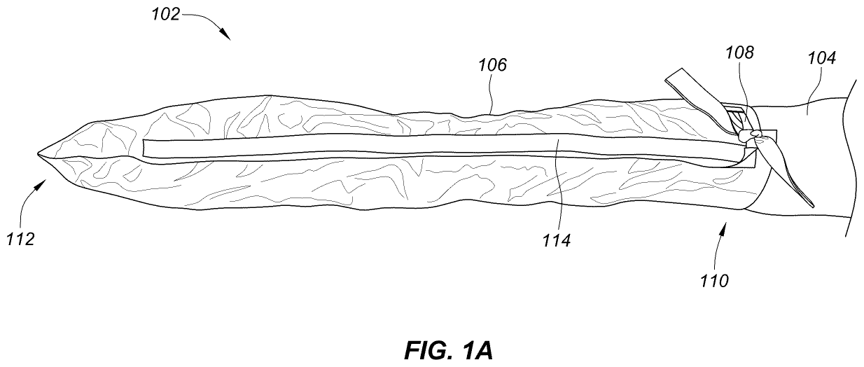

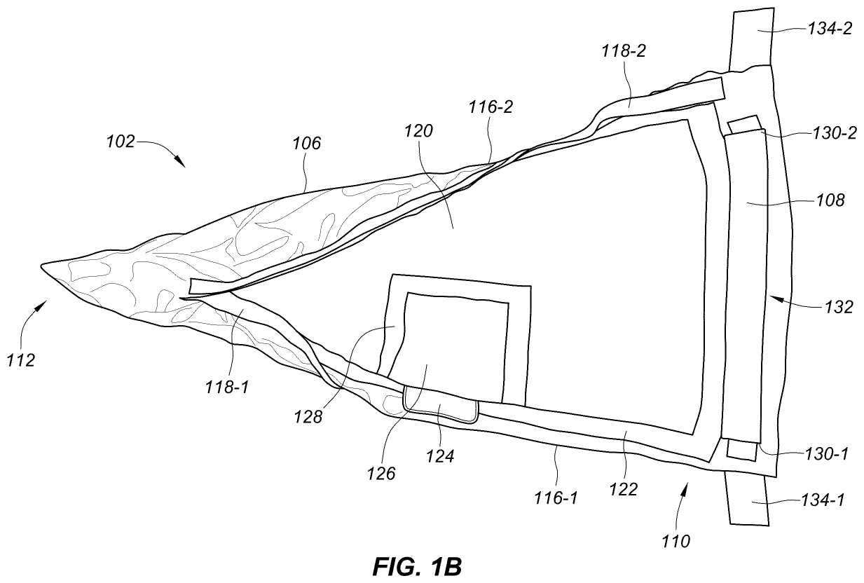

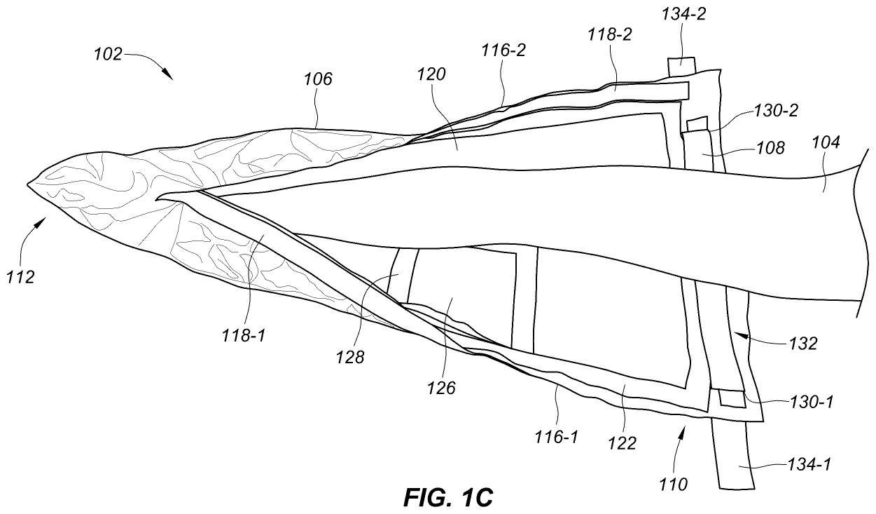

FIG. 1A is a top view of a thermal intravenous sleeve 102 disposed on the arm of a patient 104, in accordance with embodiments of the present disclosure. The thermal intravenous sleeve 102 can cover a portion of a patient's arm, thereby allowing for the patient's arm or a portion thereof to be heated. The thermal intravenous sleeve 102 can be formed from a thermally insulative material 106 allowing for heat created within the thermal intravenous sleeve 102 to be retained by the thermally insulative material 106. In some embodiments, the thermally insulative material 106 can include a particular type of reflective foil, such as mylar, tin foil, Kapton, UPILEX, and derivatives of these materials that perform the functional role of reflecting heat. In some embodiments, the thermally insulative material 106 can include an insulative foam, fabric, or other type of insulative material.

The thermal intravenous sleeve 102 can include a proximal sleeve end 110 and a distal sleeve end 112. As depicted, the thermal intravenous sleeve 102 can include a tourniquet 108 disposed at the proximal sleeve end 110. As depicted in FIG. 1A, the tourniquet 108 has been tired around the patient's arm 104. The tourniquet can be applied (e.g., tightened) on a patient's arm, before, during, and/or after heating of the patient's arm in the thermal intravenous sleeve 102.

As depicted, in some embodiments, the thermal intravenous sleeve 102 can include a scam 114 that extends between the proximal sleeve end 110 and the distal sleeve end 112. The seam 114 can include a releasable fastener, in some embodiments, that extends along the seam 114 and joins both sides of the thermal intravenous sleeve 102 together. In some embodiments, the seam 114 can extend from the proximal sleeve end 110 to the distal sleeve end 112 or somewhere therebetween. For example, as depicted in FIG. 1A, the seam 114 can extend to a portion of the thermal intravenous sleeve 102 that is located proximally in relation to the sleeve distal end 112.

As depicted, in some embodiments, the proximal sleeve end 110 of the thermal intravenous sleeve 102 can extend to a portion of a patient's arm that includes their bicep. However, in some embodiments, the thermal intravenous sleeve 102 can extend to a patient's forearm. In some embodiments, the thermal intravenous sleeve 102 can extend to a portion of a patient's arm located proximally of the patient's wrist. For example, where an intravenous needle, tube, and/or catheter is inserted into the vein in a patient's wrist, it may not be necessary for the thermal intravenous sleeve 102 to extend much past the wrist in a proximal direction.

In some embodiments, the thermal intravenous sleeve 102 can come in different diameters, which can be sized for different patients. In some embodiments, the thermal intravenous sleeve 102 can have an increased diameter from the proximal sleeve end 110 throughout a portion of the sleeve towards the distal sleeve end 112 for patients of a larger size.

FIG. 1B is a top view of the thermal intravenous sleeve 102 depicted in FIG. 1A, in an opened configuration, in accordance with embodiments of the present disclosure. In some embodiments, the thermal intravenous sleeve 102 can be formed from a thermally insulative material 106, as discussed herein. The raw thermally insulative material 106 can be fashioned into the thermal intravenous sleeve 102, as depicted in FIG. 1B. The raw thermally insulative material can have a distal end 112, a proximal end 110, a first lateral end 116-1, and a second lateral end 116-2. In some embodiments, a portion of the first lateral end 116-1 and second lateral end 116-2 can be permanently joined with one another. For example, as depicted in FIG. 1B, the distal ends of the first lateral end 116-1 and second lateral end 116-2 can be connected with one another at the distal end 112 to permanently attach the distal portions of the first lateral end 116-1 and second lateral end 116-2.

As depicted, fasteners 118-1, 118-2 can be disposed on a remainder of the first lateral end 116-1 and second lateral end 116-2 that extend proximally from the distal end 112. As discussed, the seam depicted in FIG. 1A can include a releasable fastener in some embodiments, allowing for the seam 114 to be opened or closed. In some embodiments, the fasteners 118-1, 118-2 can be hook and/or loop fasteners, such as, for example, Velcro®. In some embodiments, instead of hook and/or loop fasteners, other types of fasteners can be used, such as magnets, snaps, etc.

In some embodiments, the thermally insulative material 106 can include one or more of the same or different layers. For example, in some embodiments, the thermally insulative material 106 can be a single layer of material. In some embodiments, the thermally insulative material 106 can be more than one layer of material. For example, when the thermally insulative material 106 includes more than one layer of material, the different layers can be layered on top of one another to form the thermally insulative material. In some embodiments, the more than one layer of material can be sewn together or bonded in some way to create the effect of a single layer of material.

In some embodiments, the thermally insulative material 106 can be disposed on an exterior of the thermal intravenous sleeve 102. As depicted in FIG. 1B, a second type of material 120 can be disposed on an interior of the thermal intravenous sleeve 102. When more than one layer of material is present, the different layers of material can include different properties. For example, in some embodiments, and as depicted, the second type of material 120 can include a fabric cloth that is disposed on an interior of the thermal intravenous sleeve 102. Use of a fabric cloth can provide for a more comfortable interior of the thermal intravenous sleeve 102. Furthermore, use of a fabric cloth on the interior of the thermal intravenous sleeve 102 can include use of a fabric cloth that has wicking properties, which can provide a benefit associated with the wicking nature of cloth. For example, upon placement of the intravenous needle, tube, catheter, etc. blood can oftentimes leak from the patient. Use of a fabric cloth on the interior of the thermal intravenous sleeve can allow for blood to be absorbed by the fabric cloth, rather than pooling on an interior surface of the thermal intravenous sleeve.

As depicted in FIG. 1B, the cloth material 120 can be attached to the interior of the thermal intravenous sleeve 102. For example, as depicted in FIG. 1B, the cloth material 120 can be taped to an interior side of the thermally insulative material 106 with a tape 122. In some embodiments, the cloth material 120 can be attached to the interior of the thermally insulative material 106 with an adhesive.

As depicted in FIG. 1B, the thermal intravenous sleeve 102 can include a thermal element 124 disposed within its interior sleeve portion. In some embodiments, the thermal element 124 can be a chemically activated thermal element 124. In some embodiments, as further discussed herein, the thermal element can be an electrical heating element. The thermal element 124 can be activated by magnesium sulfate, calcium chloride, sodium acetate, among other types of catalysts. As depicted, in some embodiments, the interior sleeve portion can include a pocket 226, into which the thermal element 124 can be disposed. For example, the pocket 226 can include a piece of fabric attached at three sides to the interior sleeve portion. In some embodiments, the pocket 226 can be attached directly to the thermally insulative material 106. In some embodiments, the pocket 226 can be attached to the cloth material 120. As depicted in FIG. 1B, the pocket 226 is attached to the cloth material 120 via a taped edge 128.

As depicted in FIG. 1B, the opening of the pocket 126 can be positioned such that it faces the first lateral edge 116-1. However, in some embodiments, the opening of the pocket 126 can be positioned such that it faces the second lateral edge 116-2, the proximal sleeve portion 110, or the distal sleeve portion 112. Upon activation of the thermal element 124, heat can be generated, within the interior portion of the thermal intravenous sleeve 102, thereby causing the tissue and veins on a patient's arm to be warmed. As discussed herein, such warming can increase an effectiveness at which an intravenous needle, tube, and/or catheter can be placed in the patients' arm.

The thermal element can be located on the thermal intravenous sleeve at a location that provides heat to a particular portion of the patient's arm. For example, depending on where a needle, tube, or catheter is going to be placed in a patient's arm, the thermal element can be located accordingly. In some embodiments, the thermal element can be located towards a distal end 112 of the thermal intravenous sleeve, such that it is located over a patient's wrist, in order to warm it in preparation for intravenous insertion. In some embodiments, the thermal element can be located towards a proximal end 110 of the thermal intravenous sleeve, such that it is located over a patient's cubital fossa, in order to warm it in preparation for intravenous insertion. In some embodiments, the thermal intravenous sleeve can include a pocket disposed at the distal end 112 and a pocket disposed at a proximal end 110, such that a user can choose what pocket to put a thermal element in. For example, one pocket can be located at a portion of the thermal intravenous sleeve that aligns with a patient's wrist and one pocket can be located at a portion of the thermal intravenous sleeve that aligns with the patient's cubital fossa. In some embodiments, a pocket can be located between the proximal end 110 and the distal end 112, between portions of the thermal intravenous sleeve that align with a patient's wrist and cubital fossa. In such a configuration, a thermal intravenous single sleeve can be used for heating the wrist region and the cubital fossa region. In some embodiments that include use of an electrical heating element, two electrical heating elements can be present, one at the distal end 112 and one at the proximal end 110 and each one of the electrical heating elements can be selectively activated at the same or different times. In some embodiments that include use of an electrical heating element, the heating element can be located between portions of the thermal intravenous sleeve that correspond to the patient's wrist region and cubital fossa region.

As further depicted in FIG. 1B, the thermal intravenous sleeve 102 can include a tourniquet 108 disposed at the proximal sleeve end 110. In some embodiments, the tourniquet 108 can be formed from an elastic material (i.e., latex). In some embodiments, the tourniquet 108 can be formed from an inelastic material (i.e., nylon). As depicted, the proximal sleeve end 110 can define first and second thru-holes 130-1, 130-2, through which the tourniquet can be disposed. The first and second thru-holes 130-1, 130-2 can be longitudinal slits that extend along a longitudinal axis defined by the thermal intravenous sleeve 102. As depicted, the first and second thru-holes 130-1, 130-2 can be circumferentially spaced apart from one another about a central axis defined by the sleeve 102. For example, when the first and second lateral edges are joined with one another, the sleeve can have a tubular configuration into which the patient's arm can be inserted, thereby defining a proximal circumference about which a portion of the tourniquet 108 can extend. In some embodiments, and as depicted, the tourniquet can pass through the first and second thru-holes. In passing through the first and second thru-holes, the tourniquet 108 can be exposed to the interior portion of the thermal intravenous sleeve 102, between the first and second thru-holes 130-1, 130-2. For example, as depicted, exposed tourniquet portion 132 can be disposed between the first and second thru-holes 130-1, 130-2, allowing for most of the tourniquet to be in contact with the patient's arm. As a result, the tying ends 134-1, 134-2 of the tourniquet can be exposed, such that the tourniquet can be tightened and secured to itself, allowing for constriction on a patient's arm. Although the tourniquet 108 is depicted and discussed as a tourniquet that is tied, the tourniquet 108 can include other types of fasteners, including a hook and/or loop fastener at its ends to allow for fastening to itself.

FIG. 1C is a top and side view of a thermal intravenous sleeve 102 depicted in FIGS. 1A and 1B, in an opened configuration with the arm 104 of a patient inserted therein, in accordance with embodiments of the present disclosure. As depicted, the patient's arm 104 can be inserted into the sleeve 102, such that their bicep aligns with the tourniquet 108. Closure of the thermal intravenous sleeve 102 can include fastening the first and second lateral edges 116-1, 116-2 to one another with fasteners 118-1, 118-2 disposed on the first and second lateral edges 116-1, 116-2. For example, hook and/or loop closures 118-1, 118-2 can be disposed on each one of the first and second lateral edges 116-1, 116-2. In some embodiments, the tourniquet 108 can be tied around the patient's arm 104. Activation of the thermal element (not shown in FIG. 1C) can cause the adjacent tissue on the patient's arm and generally the interior of the thermal intravenous sleeve 102 to become heated. Thus, introduction of an intravenous needle, catheter, or tube can be more easily made into the patient's arm.

In some embodiments, although a pocket 126 is depicted as being disposed adjacent to the patient's upper forearm, one or more other pockets into which a thermal element can be inserted can be located along the patient's arm 104. For example, a pocket and corresponding thermal element can be disposed on an interior portion of the thermal intravenous sleeve that is adjacent to the patient's wrist, in some embodiments, thereby allowing for heating of the wrist and easier introduction of an intravenous needle, tube, or catheter.

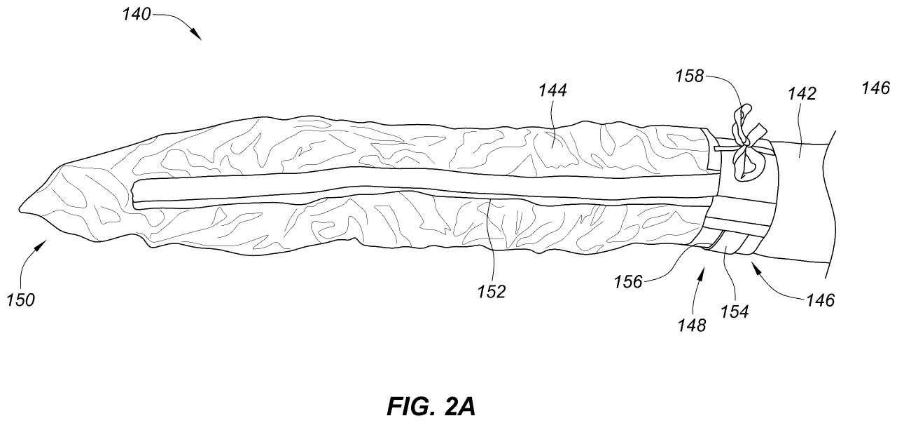

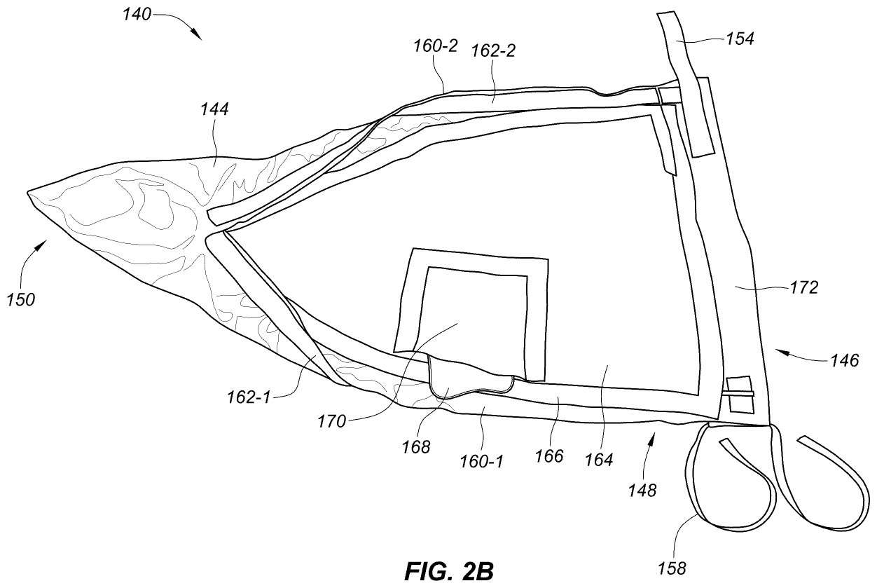

FIG. 2A is a top view of a thermal intravenous sleeve 140 with an integrated tourniquet cuff 146 disposed on the arm 142 of a patient, in accordance with embodiments of the present disclosure. The thermal intravenous sleeve can include the same or similar features as those discussed in relation to FIGS. 1A to IC, with the exception that the thermal intravenous sleeve 140 can include an integrated tourniquet cuff. For example, the thermal intravenous sleeve 140 can cover a portion of a patient's arm 142, thereby allowing for the patient's arm 142 or a portion thereof to be heated. The thermal intravenous sleeve 140 can be formed from a thermally insulative material 144 allowing for heat created within the thermal intravenous sleeve 140 to be retained by the thermally insulative material 144. In some embodiments, the thermally insulative material 144 can include a particular type of reflective foil, such as mylar, tin foil, etc. In some embodiments, the thermally insulative material 144 can include a insulative foam, fabric, or other type of insulative material.

In some embodiments, use of an integrated tourniquet cuff can allow for benefits such as a more consistent, reproducible, comfortable, and effective distribution of force versus use of an elastic tourniquet depicted in FIGS. 1A to IC. For example, an elastic tourniquet has to be manually tied and can bind/pinch the patient's skin and hair, and the elastic tourniquet can snap when being tightened.

The thermal intravenous sleeve 140 can include a proximal sleeve end 148 and a distal sleeve end 150. As depicted, the thermal intravenous sleeve 140 can include an integrated tourniquet cuff 146 disposed at the proximal sleeve end 148. As depicted in FIG. 1A, the integrated tourniquet cuff 146 has been constricted around the patient's arm 142. The integrated tourniquet cuff 146 can be applied (e.g., tightened) on a patient's arm, before, during, and/or after heating of the patient's arm in the thermal intravenous sleeve 140. As further described herein, the integrated tourniquet cuff 146 can be attached to the proximal sleeve end 148. Through attachment of the integrated tourniquet cuff 146 to the proximal sleeve end 148, the proximal sleeve end 148 can cooperate with the integrated tourniquet cuff 146 to restrict blood flow in the patient's arm 142.

In some embodiments, the integrated tourniquet cuff 146 can include a first strip of a hook and/or loop fastener 154 that extends from a second lateral end of the thermal intravenous sleeve 140 and a corresponding second strip of hook and/or loop fastener 156 that is secured to an exterior surface of the proximal sleeve end 148. Thus, upon placement of the integrated tourniquet cuff 146 on the patient's arm 142 and tightening of the integrated tourniquet cuff 146, the first and second corresponding strips of hook and/or loop fastener 154, 156 can be overlapped with one another and fastened together, thereby preventing loosening of the integrated tourniquet cuff 146. In such an embodiment, the proximal sleeve end 148 can form part of the tourniquet cuff 146. In an example, the hook and/or loop fastener can be Velcro or other re-closable fastener that functions like hook and/or loop.

In some embodiments, the integrated tourniquet cuff 146 can include a strap 158, which can include a pair of strings that can be pulled on when tightening the tourniquet cuff 146. For example, the strings can be pulled in a direction to counteract a force created when a user pulls on the hook and/or loop fastener 154 when tightening the tourniquet cuff. In the absence of the strap 158, a counteracting force to the force created when the user pulls on the hook and/or loop fastener 154 could not be created and the tourniquet cuff could freely rotate. In some embodiments, a windless strap tourniquet could be integrated in the same manner as the tourniquet cuff (154, 156) shown in FIGS. 2A and 2B.

As depicted, in some embodiments, the thermal intravenous sleeve 140 can include a seam 152 that extends between the proximal sleeve end 148 and the distal sleeve end 150. The seam 152 can include a releasable fastener, in some embodiments, that extends along the seam 152 and joins both sides of the thermal intravenous sleeve 140 together. In some embodiments, the seam 152 can extend from the proximal sleeve end 148 to the distal sleeve end 150 or somewhere therebetween. For example, as depicted in FIG. 2A, the seam 152 can extend to a portion of the thermal intravenous sleeve 140 that is located proximally in relation to the sleeve distal end 150.

As depicted, in some embodiments, the proximal sleeve end 148 of the thermal intravenous sleeve 140 can extend to the portion of a patient's arm 142 that includes their bicep. However, in some embodiments, the thermal intravenous sleeve 140 can extend to a patient's forearm. In some embodiments, the thermal intravenous sleeve 140 can extend to the portion of a patient's arm 142 located proximally of the patient's wrist. For example, where an intravenous needle, tube, and/or catheter is inserted into the vein in a patient's wrist, it may not be necessary for the thermal intravenous sleeve 140 to extend to much past the wrist in a proximal direction.

In some embodiments, the thermal intravenous sleeve 140 can come in different diameters. In some embodiments, the proximal sleeve end 148 can have different diameters, based on various sizes of patients. In some embodiments, the thermal intravenous sleeve 144 can have an increased diameter from the proximal sleeve end 148 throughout a portion of the sleeve towards the distal sleeve end 150 for larger patients.

FIG. 2B is a top view of the thermal intravenous sleeve depicted in FIG. 2A, in an opened configuration, in accordance with embodiments of the present disclosure. Embodiments depicted in FIG. 2B can include the same or similar features with respect to those embodiments discussed in relation to FIGS. 1A to IC. In some embodiments, the thermal intravenous sleeve 140 can be formed from a thermally insulative material 144, as discussed herein. The raw thermally insulative material 144 can be fashioned into the thermal intravenous sleeve 140, as depicted in FIG. 2B. The raw thermally insulative material can have a distal end 150, a proximal end 148, a first lateral end 160-1, and a second lateral end 160-2. In some embodiments, a portion of the first lateral end 160-1 and second lateral end 160-2 can be permanently joined with one another. For example, as depicted in FIG. 2B, the distal ends of the first lateral end 160-1 and second lateral end 160-2 can be connected with one another at the distal end 150 to permanently attach the distal portions of the first lateral end 160-1 and second lateral end 160-2.

As depicted, fasteners 162-1, 162-2 can be disposed on a remainder of the first lateral end 160-1 and second lateral end 160-2 that extend proximally from the distal end 150. As discussed, the seam 152 can include a releasable fastener in some embodiments, allowing for the seam 152 to be opened or closed. In some embodiments, the fasteners 162-1, 162-2 can be hook and/or loop fasteners, such as, for example, Velcro®. In some embodiments, instead of hook and/or loop fasteners, other types of fasteners can be used, such as magnets, snaps, etc.

In some embodiments, the thermally insulative material 144 can include one or more of the same or different layers. For example, in some embodiments, the thermally insulative material 144 can be a single layer of material. In some embodiments, the thermally insulative material 144 can be more than one layer of material. For example, when the thermally insulative material 144 includes more than one layer of material, the different layers can be layered on top of one another to form the thermally insulative material. In some embodiments, the more than one layer of material can be sewn together or bonded in some way to create the effect of a single layer of material.

In some embodiments, the thermally insulative material 144 can be disposed on an exterior of the thermal intravenous sleeve 140. As depicted in FIG. 2B, a second type of material 164 can be disposed on an interior of the thermal intravenous sleeve 140. When more than one layer of material is present, the different layers of material can include different properties. For example, in some embodiments, and as depicted, the second type of material 164 can include a fabric cloth that is disposed on an interior of the thermal intravenous sleeve 140. Use of a fabric cloth can provide for a more comfortable interior of the thermal intravenous sleeve 140. Furthermore, use of a fabric cloth on the interior of the thermal intravenous sleeve 140 can provide a benefit associated with the wicking nature of cloth. For example, upon placement of the intravenous needle, tube, catheter, etc. blood can oftentimes leak from the patient. Use of a fabric cloth on the interior of the thermal intravenous sleeve can allow for blood to be absorbed by the fabric cloth, rather than pool.

As depicted in FIG. 1B, the cloth material 164 can be attached to the interior of the thermal intravenous sleeve 140. For example, as depicted in FIG. 2B, the cloth material 164 can be taped to an interior side of the thermally insulative material 144 with a tape 166. In some embodiments, the cloth material 164 can be attached to the interior of the thermally insulative material 144 with an adhesive.

As depicted in FIG. 2B, the thermal intravenous sleeve 140 can include a thermal element 168 disposed within its interior sleeve portion, as further discussed herein, for example, in relation to FIGS. 1A to IC. In some embodiments, the thermal element 168 can be disposed in a pocket 170.

As further depicted in FIG. 2B, the thermal intravenous sleeve 140 can include an integrated tourniquet cuff 146 disposed at the proximal sleeve end 148. In some embodiments, the integrated tourniquet cuff 146 can include a cuff portion 172 formed from a combination of the proximal sleeve end 148 and first and second corresponding fasteners 154, 156. For example, as discussed in relation to FIG. 2A, the integrated tourniquet cuff 146 can include a first strip of a hook and/or loop fastener 154 that extends from a second lateral end 160-2 of the thermal intravenous sleeve 140 and a corresponding second strip of hook and/or loop fastener 156 that is secured to an exterior surface of the proximal sleeve end 148 (hidden from view in FIG. 2B, but on the back side of the cuff portion 172 and further depicted in FIG. 2A). Thus, upon placement of the tourniquet cuff on the patient's arm 142 and tightening of the integrated tourniquet cuff 146, the first and second corresponding strips of hook and/or loop fastener 154, 156 can be overlapped with one another and fastened together, thereby preventing loosening of the integrated tourniquet cuff 146. For example, the first strip of hook and/or loop fastener 154 can be overlapped with the exterior surface of the proximal sleeve end 148 and the corresponding second strip of hook and/or loop fastener, as further depicted in FIG. 2A. In some embodiments, use of an integrated tourniquet cuff may increase an case of operation of the thermal intravenous sleeve 140, reducing the need to separately manipulate a tourniquet cuff in relation to the thermal intravenous sleeve 140. In some embodiments, case of use can be increased as a result of the use of hook and/or loop fasteners. For example, the use of hook and/or loop fasteners may alleviate the need to tie a tourniquet cuff.

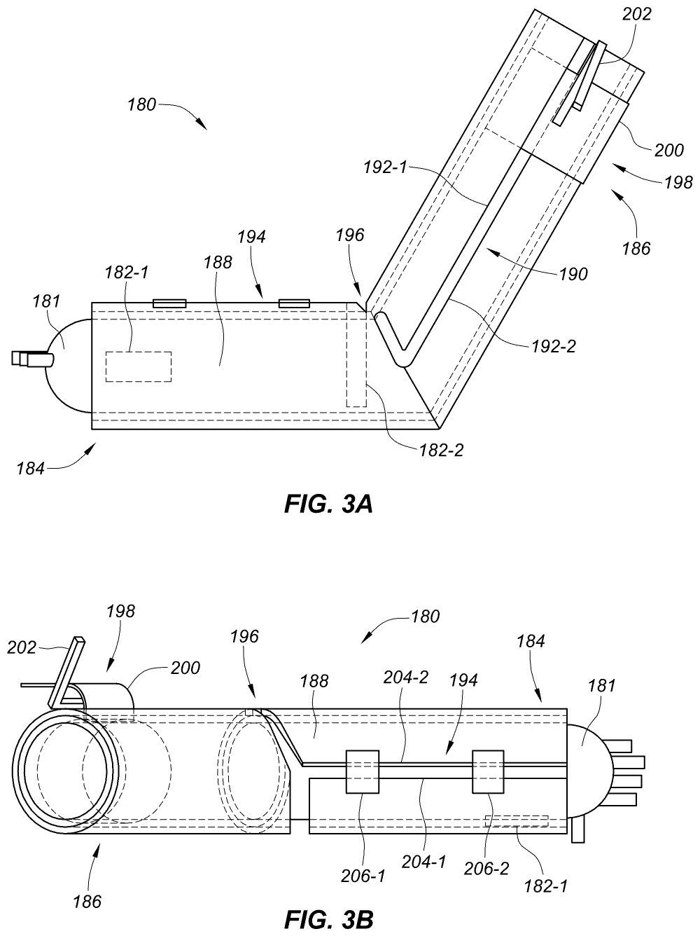

FIG. 3A is a side view of a thermal intravenous sleeve 180 that includes an electrical heating element 182-1, 182-1, in accordance with embodiments of the present disclosure. In some embodiments, the thermal intravenous sleeve 180 can include a distal sleeve end 184 and a proximal sleeve end 186 and can be disposed on a patient's arm 181, which is depicted by a modeled representation in FIG. 3A. As discussed herein, the thermal intravenous sleeve 180 can be formed from a thermally insulative material 188. In some embodiments, the thermally insulative material 188 can include material such as that discussed in relation to FIGS. 1A to 2B, for example, the thermally insulative material 188 can be a reflective type of insulative material. In some embodiments, a reflective type of insulative material can provide a good thermal efficiency in relation to other types of insulative materials, which can make it a suitable choice for use with a chemically activated thermal element. In some embodiments that employ one or more electrical heating elements 182-1, 182-2, a thermal efficiency of the insulative material may not be as important, since the amount and duration of which heat can be applied is not limited by a chemical process. Accordingly, in some embodiments, while the thermally insulative material 188 can include a reflective type of material, the thermally insulative material 188 can include a neoprene, foam, woven cloth, among other types of thermally insulative materials 188. For example, one important aspect associated with a thermally insulative material 188 can be the fact that it can be pliable to some degree, such that it can allow the one or more electrical heating elements to be in thermal contact with the patient's skin.

In some embodiments, the thermally insulative material 188 can define a tube into which a patient's arm can be inserted. In some embodiments, the tube can include one or more seams that extend along a longitudinal axis that is defined by the thermally insulative sleeve. For example, as depicted in FIG. 3A, a seam 190 can extend along an upper portion of the thermal intravenous sleeve 180. The seam 190 can be formed as a result of the relative positioning of a first lateral end 192-1 and second lateral end 192-2 of the upper portion of the thermal intravenous sleeve 180, with respect to one another.

As further depicted, the thermal intravenous sleeve 180 can include a lower portion that also includes a seam 194, as further depicted in FIG. 3A. In some embodiments, a flexible joint 196 can be included between the upper portion and lower portion of the thermal intravenous sleeve 180. For example, as depicted, a gap can be defined between the upper portion and lower portion of the thermal intravenous sleeve 180. In some embodiments, the gap can be defined about a portion of a circumference of the thermal intravenous sleeve 180, as depicted in FIG. 3A. For instance, the gap can be defined on the portion of the thermal intravenous sleeve 180 that would be disposed about the cubital fossa region of the patient. In some embodiments, a flexible fabric can generally be disposed about the flexible joint 196 to allow a patient or physician to manipulate the patient's arm.

As depicted, a first electrical heating element 182-1 can be disposed in a distal sleeve end 184. In some embodiments, the first electrical heating element 182-1 can be disposed in a portion of the distal sleeve end 184, such that the first electrical heating element can be positioned so that it can contact one or more veins in the patient's wrist. As further depicted, a second electrical heating element 182-2 can be disposed in a more proximal portion of the lower portion of the thermal intravenous sleeve 180. In some embodiments, the second electrical heating element 182-2 can be disposed in a proximal portion of the lower portion of the thermal intravenous sleeve 180, such that the second electrical heating element can be positioned so that it can contact one or more veins in the patient's forearm. As discussed herein, in some embodiments, the thermal intravenous sleeve 180 can include a single electrical heating element disposed between the regions of the sleeve that correspond to a patient's wrist and cubital fossa.

In some embodiments, although first and second electrical heating elements are shown as being disposed in the thermal intravenous sleeve 180, less than two electrical heating elements or greater than two electrical heating elements can be disposed in the thermal intravenous sleeve portion 180. In some embodiments, one or more electrical leads can be connected with the one or more electrical heating elements 182-1, 182-2. In some embodiments, the electrical heating elements 182-1, 182-2 can be controlled together and/or in some embodiments, the electrical heating elements 182-1, 182-2 can be controlled separately. In an example, if a physician wishes to place a needle, tube, catheter, etc. in a particular portion of a patient's arm, the physician can activate only the heating elements that are associated with that portion of the patient's arm. For example, if a physician wishes to introduce a needle, tube, catheter, etc. into a patient's wrist, then the physician can only activate the electrical heating element 182-1. In some embodiments, a thermostat can be connected to one or more of the electrical heating elements to provide control over the temperature of one or more of the electrical heating elements 182-1, 182-2 individually or in combination with one another. In some embodiments, the one or more heating elements 182-1, 182-2 can be controlled by a thermostat, which can provide for on/off functionality, temperature control, as well as a timer to control the duration over which electricity is provided to the electrical heating elements 182-1, 182-2.

As depicted, in some embodiments, the first electrical heating element 182-1 can extend in a longitudinal direction by a greater distance than in a lateral direction, as depicted. However, in some embodiments, the first electrical heating element 182-1 can extend in the longitudinal and lateral directions by the same distance and/or can extend further in the lateral direction than in the longitudinal direction. As further depicted, the second electrical heating element 182-2 can extend in the longitudinal direction by a lesser distance than in the lateral direction. However, in some embodiments, the second electrical heating element 182-2 can extend in the longitudinal and lateral directions by the same distance and/or can extend further in the longitudinal direction than in the lateral direction.

In some embodiments, the electrical heating elements can be disposed on an interior surface of the thermal intravenous sleeve 180. For example, the electrical heating elements can be disposed adjacent to a patient's skin. In some embodiments, the electrical heating elements can be disposed beneath a layer of material, such that the layer of material separates the patient's skin from the heating element, yet allows the electrical heating element to be in thermal contact with the patient's skin. In some embodiments, the electrical heating element can extend inward in a radial direction from the interior surface of the thermal intravenous sleeve 180, such that an inwardly disposed bump is present where the electrical heating element is located. In some embodiments, this can allow for an impression to be formed on the patient's skin, which can indicate to a physician, where the electrical heating element had been placed. In accordance with embodiments of the present disclosure, this can provide a visually identifiable target area where the physician can try and access an underlying vein. In some embodiments, a pattern can be disposed on a raised portion and/or the electrical heating element, such that a corresponding pattern is disposed on the skin and can be easily identified as a target area for intravenous insertion of a needle, tube, and/or catheter. In an example, the corresponding pattern can include a number of dots, and/or discrete raised portions, such that each dot and/or discreet raised portion forms an individual impression in the patient's skin, thereby helping a physician identify where the electrical heating element had been placed.

In some embodiments, the proximal sleeve end 186 can include an integrated tourniquet cuff 198, which can include the same or similar features as those discussed in relation to the integrated tourniquet cuff 198. In an example, the tourniquet cuff 200 can include a corresponding pair of hook and/or loop fasteners. For example, a first hook and/or loop fastener can extend from a first lateral edge 192-1 and can extend laterally away from the lateral edge, such that it has sufficient length to be wrapped around a portion of the patient's arm and overlap a corresponding hook and/or loop fastener disposed on an exterior surface of the proximal sleeve portion 186. The integrated tourniquet cuff 198 can further include a windlass strap 202, which can allow for more minute adjustment of tension applied to the patient's arm with the tourniquet cuff.

FIG. 3B is a top view of the thermal intravenous sleeve 180 depicted in FIG. 3A, in accordance with embodiments of the present disclosure. As depicted, the thermal intravenous sleeve 180 includes a distal sleeve end 184 and a proximal sleeve end 186 and can be formed from a thermally insulative material 188. As depicted, the thermal intravenous sleeve 180 can include an electrical heating element 182-1, which can be disposed within an interior portion of the thermal intravenous sleeve 180, such that the electrical heating element 182-1 is in thermal contact with a patient's skin.

In some embodiments, the lower portion of the thermal intravenous sleeve 180 can include a first lateral edge 204-1 and a second lateral edge 204-2. In some embodiments, the lateral edges 204-1, 204-2 can be separated from one another, such that a seam 194 is separated, thereby allowing room for a patient to insert their arm into the thermal intravenous sleeve 180. As depicted, the thermal intravenous sleeve 180 can include one or more fasteners 206-1, 206-1 disposed at an interface of the first and second lateral edges 204-1, 204-2, along the seam 194. The fasteners 206-1, 206-2 can be hook and/or loop fasteners in some embodiments and can be used to hold the lower portion of the thermal intravenous sleeve 180 in a “closed” state, such that the thermal intravenous sleeve 180 can remain stationary on a patient's arm, if the fasteners 206-1, 206-2 are tightened accordingly. In some embodiments, the upper portion of the thermal intravenous sleeve 180 can also include fasteners to maintain a desired spacing between first and second lateral edges 192-1, 192-2.

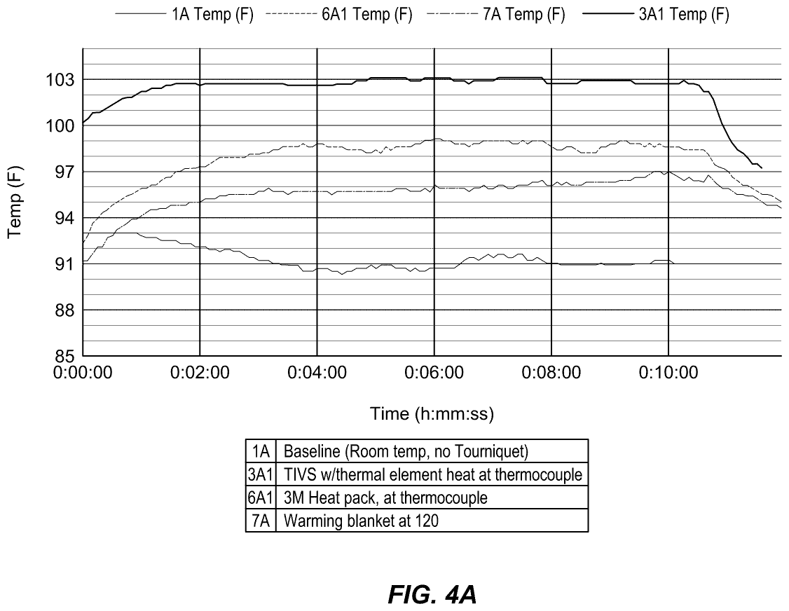

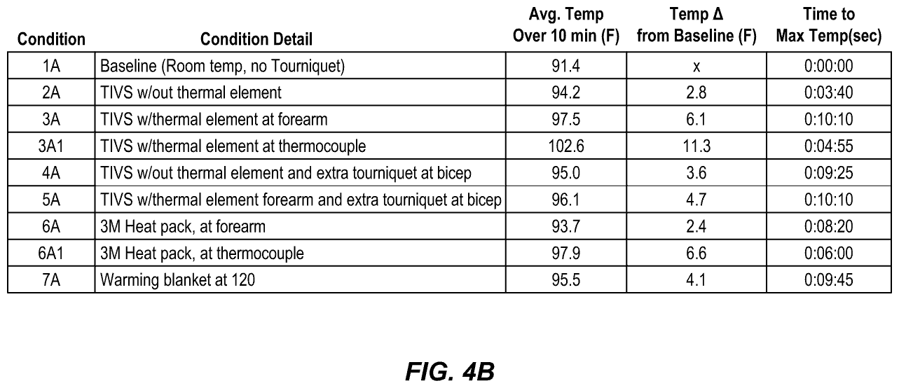

FIG. 4A depicts a graph depicting test results associated with embodiments of the present disclosure versus known methods, in accordance with embodiments of the present disclosure. Experimental tests were conducted, using a thermal intravenous sleeve (TIVS) with a thermal element in accordance with embodiments of the present disclosure to warm a patient's arm. Further experimental tests were conducted using other methods known in the art to heat the patient's arm and the resulting data was charted in the graph depicted in FIG. 4A, which represents temperature versus time. To collect temperature data, a thermocouple was placed on the patient's arm in the cubital fossa region. With respect to test 1A, baseline data was collected using a thermocouple placed on a patient's arm near the cubital fossa region. Data was collected from the thermocouple for a period of time exceeding 10 minutes and mapped on the graph depicted in FIG. 4A. With respect to test 3A1, embodiments of the present disclosure were used to evaluate their efficacy in warming a patient's tissue. Data was collected using a thermocouple placed on a patient's arm near the cubital fossa. A thermal intravenous sleeve, as discussed herein, was placed on the patient's arm with a thermal element in the form of a chemical heat pack being located on top of the thermocouple. Data was collected from the thermocouple for a period of time exceeding 10 minutes and mapped on the graph depicted in FIG. 4A. With respect to test 6A1, a thermocouple was placed on the patient's arm near the cubital fossa and a heat pack (e.g., 3M heat pack) was placed on top of the thermocouple. Data was collected from the thermocouple for a period of time exceeding 10 minutes and mapped on the graph depicted in FIG. 4A. With respect to test 7A, a thermocouple was placed on the patient's arm near the cubital fossa and a warming blanket set to 120 degrees Fahrenheit was placed on top of the thermocouple. Data was collected from the thermocouple for a period of time exceeding 10 minutes and mapped on the graph depicted in FIG. 4A. As can be seen in FIG. 4A, use of the thermal intravenous sleeve can result in a higher attained temperature at the thermocouple than other known methods, which can allow for an easier intravenous insertion of a needle, tube, and/or catheter, for example.

FIG. 4B depicts a table depicting test results associated with embodiments of the present disclosure versus known methods, in accordance with embodiments of the present disclosure. In the listed tests, a thermocouple placed near the cubital fossa was used to obtain temperature data. In test 1A, a baseline temperature was taken at room temperature using no tourniquet cuff and no heating element. In test 2A, embodiments of the present disclosure were used without a heating element. For example, a thermal intravenous sleeve was placed on a patient's arm without a thermal element inside of the thermal intravenous sleeve. In test 3A, a thermal intravenous sleeve in accordance with embodiments of the present disclosure was placed on a patient's arm and a thermal element in the form of a chemical heat pack was placed on the patient's forearm, at a position distal to the thermocouple. In test 3A, a thermal intravenous sleeve in accordance with embodiments of the present disclosure was placed on a patient's arm and a thermal element in the form of a chemical heat pack was placed on top of the thermocouple. In test 4A, a thermal intravenous sleeve in accordance with embodiments of the present disclosure was placed on a patient's arm without a thermal element and an extra tourniquet was placed at the patient's bicep. In test 5A, a thermal intravenous sleeve in accordance with embodiments of the present disclosure was placed on a patient's arm with a thermal element in the form of a chemical heat pack located on the patient's forearm and an extra tourniquet was placed at the patient's bicep. In test 6A, a chemical heat pack was placed on the patient's forearm and in test 6A1, the chemical heat pack was placed on top of the thermocouple. In test 7A, a warming blanket heated to 120 degrees Fahrenheit was placed on the patient's arm, over the thermocouple. As can be seen in the test results, embodiments of the present disclosure can provide the greatest average temperature over a duration of 10 minutes and the greatest change in temperature from baseline. Additionally, as can be seen in the test results, embodiments of the present disclosure can provide the fastest time to max temp, thereby allowing for an increased speed at which a needle, tube, and/or catheter can be placed in a patient.

Embodiments are described herein of various apparatuses, systems, and/or methods. Numerous specific details are set forth to provide a thorough understanding of the overall structure, function, manufacture, and use of the embodiments as described in the specification and illustrated in the accompanying drawings. It will be understood by those skilled in the art, however, that the embodiments may be practiced without such specific details. In other instances, well-known operations, components, and elements have not been described in detail so as not to obscure the embodiments described in the specification. Those of ordinary skill in the art will understand that the embodiments described and illustrated herein are non-limiting examples, and thus it may be appreciated that the specific structural and functional details disclosed herein may be representative and do not necessarily limit the scope of the embodiments, the scope of which is defined solely by the appended claims.

Reference throughout the specification to “various embodiments,” “some embodiments,” “one embodiment,” or “an embodiment”, or the like, means that a particular feature, structure, or characteristic described in connection with the embodiment(s) is included in at least one embodiment. Thus, appearances of the phrases “in various embodiments,” “in some embodiments,” “in one embodiment,” or “in an embodiment,” or the like, in places throughout the specification, are not necessarily all referring to the same embodiment. Furthermore, the particular features, structures, or characteristics may be combined in any suitable manner in one or more embodiments. Thus, the particular features, structures, or characteristics illustrated or described in connection with one embodiment may be combined, in whole or in part, with the features, structures, or characteristics of one or more other embodiments without limitation given that such combination is not illogical or non-functional.

Although at least one embodiment for a thermal intravenous sleeve has been described above with a certain degree of particularity, those skilled in the art could make numerous alterations to the disclosed embodiments without departing from the spirit or scope of this disclosure. All directional references (e.g., upper, lower, upward, downward, left, right, leftward, rightward, top, bottom, above, below, vertical, horizontal, clockwise, and counterclockwise) are only used for identification purposes to aid the reader's understanding of the present disclosure, and do not create limitations, particularly as to the position, orientation, or use of the devices. Joinder references (e.g., affixed, attached, coupled, connected, and the like) are to be construed broadly and may include intermediate members between a connection of elements and relative movement between elements. As such, joinder references do not necessarily infer that two elements are directly connected and in fixed relationship to each other. It is intended that all matter contained in the above description or shown in the accompanying drawings shall be interpreted as illustrative only and not limiting. Changes in detail or structure may be made without departing from the spirit of the disclosure as defined in the appended claims.

Any patent, publication, or other disclosure material, in whole or in part, that is said to be incorporated by reference herein is incorporated herein only to the extent that the incorporated materials does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

What is claimed:

1. A thermal intravenous sleeve, comprising:
a thermally insulative material, wherein the thermally insulative material includes a proximal end, a distal end, first lateral end, and second lateral end, wherein a portion of the first lateral end and the second lateral end are joined, thereby defining an interior sleeve portion and an exterior sleeve portion;
a thermal element disposed within the interior sleeve portion; and
a tourniquet disposed at a proximal end of the thermal intravenous sleeve.

2. The thermal intravenous sleeve of claim 1, further comprising a pocket disposed on the interior sleeve portion into which the thermal element is disposed.

3. The thermal intravenous sleeve of claim 2, wherein the thermal element includes a chemical heating element disposed within the pocket.

4. The thermal intravenous sleeve of claim 1, wherein the thermal element includes an electrical heating element.

5. The thermal intravenous sleeve of claim 1, wherein the thermally insulative material includes a heat reflective foil.

6. The thermal intravenous sleeve of claim 5, further comprising a cloth material disposed on the interior sleeve portion.

7. The thermal intravenous sleeve of claim 1, wherein the first lateral end and the second lateral end are releasably joined to one another.

8. The thermal intravenous sleeve of claim 7, wherein the first lateral end and the second lateral end are releasably joined to one another with a hook and loop fastener.

9. The thermal intravenous sleeve of claim 1, wherein the tourniquet is an integrated tourniquet cuff attached to the proximal end of the intravenous sleeve.

10. The thermal intravenous sleeve of claim 1, wherein the thermal intravenous sleeve defines first and second thru-holes at the proximal end of the intravenous sleeve.

11. The thermal intravenous sleeve of claim 10, wherein the first and second thru-holes are circumferentially spaced apart from one another about a central axis defined by the sleeve.

12. The thermal intravenous sleeve of claim 11, wherein the tourniquet passes through the first and second thru-holes.

13. The thermal intravenous sleeve of claim 12, wherein the tourniquet is exposed to the interior sleeve portion between a first circumference extending between the first and second thru-hole.

14. The thermal intravenous sleeve of claim 13, wherein the tourniquet is exposed to the exterior sleeve portion between a second circumference extending between the first and second thru-hole.

15. The thermal intravenous sleeve of claim 13, wherein the first circumference and the second circumference together define an opening to the proximal end of the intravenous sleeve.

16. A thermal intravenous sleeve, comprising:
a thermally insulative material, wherein the thermally insulative material includes a proximal end, a distal end, first lateral end, and second lateral end, wherein a portion of the first lateral end and the second lateral end are joined, thereby defining an interior sleeve portion and an exterior sleeve portion;
an electrical heating element disposed within the interior sleeve portion; and
an integrated tourniquet cuff disposed at a proximal end of the thermal intravenous sleeve.

17. The thermal intravenous sleeve of claim 1, wherein the thermal intravenous sleeve includes a first and second heating element, wherein:
the first heating element is disposed on the interior sleeve portion in an area that corresponds to a patient's wrist, and
the second heating element is disposed on the interior sleeve portion in an area that corresponds to the patient's cubital fossa.

18. The thermal intravenous sleeve of claim 17, wherein a temperature of the first heating element and the second heating element are independently controllable.

19. A thermal intravenous sleeve, comprising:
a thermally insulative material, wherein the thermally insulative material includes a proximal end, a distal end, first lateral end, and second lateral end, wherein a portion of the first lateral end and the second lateral end are joined, thereby defining an interior sleeve portion and an exterior sleeve portion;
a pocket disposed on the interior sleeve portion;
a chemical heating element disposed in the pocket; and
an tourniquet disposed at a proximal end of the thermal intravenous sleeve.

20. The thermal intravenous sleeve of claim 19, wherein the pocket is disposed on the interior sleeve portion in an area that corresponds to a patient's cubital fossa.
